(19) United States
(12) Patent Application Publication
(10) Pub. No.: US 2008/0279843 A1
(43) Pub. Date: Nov. 13, 2008

(54) Method For Improving Insulin Sensitivity By Administering an Inhibitor of Antitrypsin

(75) Inventor: Zoltan Kiss, Austin, TX (US)

Correspondence Address: FAEGRE & BENSON LLP;PATENT DOCKETING, 2200 WELLS FARGO CENTER, 90 SOUTH SEVENTH STREET, MINNEAPOLIS, MN, 55402-3901, US

(73) Assignee: Essential SkinCare, LLC, Austin, MN (US)

(21) Appl. No.: 10/582,391

(22) PCT Filed: Dec. 9, 2004
(86) PCT No.: PCT/US04/41508

§ 371 Date: Jun. 9, 2006

Related U.S. Application Data
(60) Provisional application No. 60/528,017, filed on Dec. 9, 2003.

(51) Int. Cl. A61K 38/46; A61K 31/192; A61K 38/28; A61P 3/10; A61K 31/60; A61K 31/426

(52) U.S. Cl. 424/94.6; 514/568; 514/3; 514/369; 514/165

(57) ABSTRACT

Methods of delaying the onset or treating the progression of Type 2 diabetes in subjects that have increased blood levels of an inflammation marker protein. In one embodiment, the method includes administering an inhibitor OF α₁-antitrypsin (AT) to the subject, and optionally co-administering an anti-diabetic medicament or anti-inflammatory agent to the subject. Treatment regimens provided by the invention may be used to delay the onset of or to control Type 2 diabetes. The invention provides for the use of an inhibitor of α₁-antitrypsin, such as gemfibrozil (GF), for the manufacture of a medicament for the uses described herein. The invention further provides combinations of agents for treating or delaying the progression or onset of diabetes, comprising an inhibitor of α₁-antitrypsin and an anti-inflammatory agent and/or an anti-diabetic medicament.

### TECHNICAL FIELD

Significant recent changes in human behavior and lifestyle as well as the human environment have resulted in the escalation of diabetes during the last decades. Diabetes is a disease characterized by elevated levels of blood plasma glucose, or hyperglycemia. Hyperglycemia, if uncontrolled, can lead to other complications, such as blindness, kidney disease, heart disease, stroke, nerve diseases, circulatory disorders, and impotence in males. Diabetes is a chronic disease with diverse pathologic manifestations, and is accompanied by lipid metabolism and cardiovascular disorders as well as glycometabolism disorders.

Diabetes already afflicts an estimated 6% of the adult population in Western society, and its worldwide frequency is projected to grow by 6% per annum, potentially reaching a total of 200-300 million cases in 2010 [Zimmet, P., Alberti, K. G. M. and Shaw, J., “Global and societal implications of the diabetes epidemic.” Nature 414, 782-787 (2001)]. The main forces driving this increasing incidence are sedentary lifestyle and a staggering increase in obesity.

Despite large variations in carbohydrate intake with various meals, blood glucose normally remains in a narrow range between 4 and 7 mM in non-diabetic individuals. Such tight control is regulated by the balance among three major mechanisms, i.e. (i) glucose absorption from the intestine, (ii) glucose production by the liver, and (iii) uptake and metabolism of glucose by the peripheral tissues, mainly the skeletal muscle and fat tissue. In skeletal muscle and fat tissue, insulin increases the uptake of glucose, increases the conversion of glucose to glycogen, and increases conversion of glucose to fat (mainly triglycerides). In the liver, insulin inhibits the release of glucose from glycogen. Insulin is the only known hormone which can regulate all three mechanisms required to maintain the blood glucose level in the normal range [Saltiel, A. R. and Kahn, C. R., “Insulin signaling and the regulation of glucose and lipid metabolism.” Nature 414, 799-806 (2001)].

Diabetes mellitus is a heterogeneous group of disorders characterized by high blood glucose (sugar) levels. There are two main types of diabetes. Type 1, or insulin-dependent diabetes, results from a deficiency of insulin due to autoimmunological destruction of the insulin-producing pancreatic β-cell islets [Bell, G. I. and Polonsky, K. S., “Diabetes mellitus and genetically programmed defects in β-cell function.” Nature 414, 788-791 (2001); Mathis, D., Vence, L. and Benoist, C., “β-cell death during progression to diabetes.” Nature 414, 792-798 (2001)]. People with Type 1 diabetes must take exogenous insulin for survival to prevent the development of ketoacidosis.

In Type 2 diabetes, or non-insulin-dependent diabetes mellitus (NIDDM), muscle, fat, and liver cells are resistant to the actions of insulin. Furthermore, compensatory mechanisms that are activated in β-cells to secrete more insulin to maintain blood glucose levels within a normal physiological range fail to function properly. Type 2 diabetes accounts for about 90% of all diabetes [Saltiel, A. R., “New perspectives into the molecular pathogenesis and treatment of Type 2 diabetes.” Cell 104, 517-529 (2001)]. Type 2 diabetics are often prescribed blood glucose-lowering sulfonylurea-based or -derived drugs, which are associated with the stimulation of insulin production in the pancreatic β-cells. Alternatively, patients suffering from Type 2 diabetes may also be prescribed biguanide-based or -derived drugs, which are associated with increasing a patient's sensitivity to insulin. Inhibitors of α-glucosidase, which decrease absorption of glucose from the intestine, may also be prescribed. Finally, thiazolidinediones, may be prescribed.

Diabetes is a potentially very dangerous disease because it is associated with markedly increased incidence of coronary, cerebral, and peripheral artery disease. As a result, patients with diabetes have a much higher risk of myocardial infarction, stroke, limb amputation, renal failure, or blindness. Atherosclerotic cardiovascular disease is responsible for 80% of diabetic mortality and more than 75% of all hospitalizations for diabetic complications [Moller, D. E., “New drug targets for Type 2 diabetes and the metabolic syndrome.” Nature 414, 821-827 (2001)]. Recent evidence indicate that hyperglycemia leads to overproduction of superoxide accounting for vascular damage, which, in turn, underlies most diabetic complications [Brownlee, M., “Biochemistry and molecular cell biology of diabetic complications.” Nature 414, 813-820 (2001); Ho, E. and Bray, T. M., “Antioxidants, NFκB activation, and diabetogenesis.” Proc. Soc. Exp. Biol. Med. 222, 205-213 (1999)].

Recently, a lot of effort has been made to determine the factors that contribute to diabetes. It is clear that these factors include free fatty acids (FFAs), particularly the saturated species. In the liver, FFAs cause insulin resistance by inhibiting insulin suppression of glycogenolysis resulting in increased glucose output [Boden, G., Cheung, P., Stein, T. P., Kresge, K. and Mozzoli, M., “FFA cause hepatic insulin resistance by inhibiting suppression of glycogenolysis.” Am. J. Physiol. Endocrinol. Metab. 283, E-12-E19 (2001)]. In muscle, FFAs decrease insulin sensitivity by inhibiting phosphatidylinositol 3-kinase activity, a downstream effector of insulin action [Yu, C., Chen, Y., Cline, G. W., Zhang, D., Zong, H., Wang, Y., Bergeron, R., Kim, M. F., Cushman, S. W., Cooney, G. J., Atcheson, B., White, M. F., Kraegen, E. W. and Shulman, G. I., “Mechanism by which fatty acids inhibit insulin activation of insulin receptor substrate-1 (IRS-1)-associated phosphatidylinositol 3-kinase in Muscle.” J. Biol. Chem. 277, 50230-50236 (2002)]. Finally, in the pancreas, saturated FFAs induce apoptotic death of β-cells [Maedler, K., Spinas, G. A., Dyntar, D., Moritz, W., Kaiser, N. and Donath, M. Y. “Distinct effects of saturated and monounsaturated fatty acids on β-cell turnover and function.” Diabetes 50, 69-76 (2001)].

Another risk factor for developing Type 2 diabetes is inflammation, including subchronic inflammation, which is accompanied by the synthesis of liver-derived acute phase proteins such as C-reactive protein, α₁-antitrypsin, serum amyloid, haptoglobin, fibrinogen, α₁-acid glycoprotein, and α₁-antichymotrypsin. Of the currently recognized mediators of inflammation (tumor necrosis factor, interleukin-1, and interleukin-6) only interleukin-6 is capable of eliciting the full range of acute phase protein changes seen in inflammation. Of the inflammation markers, interleukin-6, along with C-reactive protein, has definitely been identified as a risk factor for developing Type 2 diabetes [Pradhan, A. D., Manson, J. E., Rifai, N., Buring, J. E. and Ridker, P. M. “C-reactive protein, interleukin 6, and risk of developing Type 2 diabetes mellitus. JAMA 286, 327-334 (2001)]. The mechanism by which inflammation modulates insulin sensitivity is presently not clear. However, it is clear that insulin is capable of down-regulating cytokine-stimulated expression of acute phase protein in liver cells [Thompson, D., Harrison, S. P., Evans, S. W. and Whicher, J. T. “Insulin modulation of acute-phase protein production in a human hepatoma cell line. “Cytokine 3, 619-626 (1991); Campos, S. P. and Baumann, H. “Insulin is a prominent modulator of the cytokine-stimulated expression of acute-phase plasma protein genes.” Mol. Cell. Biol. 12, 1789-1797 (1992)].

Since interleukin-6 also regulates hepatic synthesis of AT, one would expect that increases in blood AT level, as part of the acute phase response, will also be indicative of risk for developing Type 2 diabetes. Indeed, several laboratories consistently found that higher than normal blood concentrations of AT, along with other acute phase proteins, predict Type 2 diabetes [Ganrot, P. O, Gydell, K. and Ekelund, H. “Serum concentration of α₂-macroglobulin, haptoglobin and α₁-antitrypsin in diabetes mellitus.” Acta Endocrinologica 55, 537-544 (1967); McMillan, D. E. “Increased levels of acute-phase serum proteins in diabetes.” Metabolism 38, 1042-1046 (1989); Schmidt, M. I., Duncan, B. B., Sharrett, A. R., Lindberg, G., Savage, P. J., Offenbacher, S., Azambuja, M. I., Tracy, R. P. and Heiss, G. “Markers of inflammation and prediction of diabetes mellitus in adults.” The Lancet 353, 1649-1652 (1999)]. However, it is not known whether the relationship between AT level and diabetes is causal or not.

AT belongs to the large family of serine protease inhibitors, or “serpins,” that act as irreversible suicide inhibitors of proteases [Janciauskiene, S. “Conformational properties of serine proteinase inhibitors (serpins) confer multiple pathophysiological roles.” Biochim. Biophys. Acta 1535, 221-235 (2001)]. AT is a particularly effective inhibitor of elastase, but is also inhibits other proteases such as trypsin. AT deficiency, often caused by its oxidative damage in smokers, is causally related to emphysema due to the uncontrolled action of proteases in the lung. AT deficiency may be reversed by replacement therapy with purified AT [Wewers, M. D., Casolaro, M. A., Sellers, S. E., Swayze, S. C., McPhaul, K. M., Wittes, J. T. and Crystal, R. G. “Replacement therapy for alpha₁-antitrypsin deficiency associated with emphysema.” N. Engl. J. Med. 316, 1055-1062 (1987)].

AT has several biological actions that may or may not relate to its ability to inhibit proteases. Thus, AT has been shown to both stimulate and inhibit proliferation of various cell types [Perraud, F., Besnard, F., Labourdette, G. and Sensenbrenner, M. “Proliferation of rat astrocytes, but not of oligodendrocytes, is stimulated in vitro by protease inhibitors.” Int. J. Devl. Neuroscience 6, 261-266 (1988); She, Q.-B., Mukherjee, J. J., Crilly, K. S. and Kiss, Z. “α₁-antitrypsin can increase insulin-induced mitogenesis in various fibroblast and epithelial cell lines.” FEBS Lett. 473, 33-36 (2000); Dabbagh, K., Laurent, G. J., Shock, A., Leoni, P., Papakrivopoulou, J. and Chambers, R. C. “Alpha-1-antitrypsin stimulates fibroblast proliferation and procollagen production and activates classical MAP kinase signaling pathways.” J. Cell. Physiol. 186, 73-81 (2001); Graziadei, I., Gaggl, S., Kaserbacher, R., Braunsteiner, H. and Vogel, W. “The acute phase protein α₁-antitrypsin inhibits growth and proliferation of human early erythroid progenitor cells (burst-forming units-erythroid) and of human erythroleukemic cells (K562) in vitro by interfering with transferring iron uptake.” Blood 83, 260-268 (1994)]. AT also was shown to block the release of transforming growth factor-α from MCF-7 human breast cancer cells resulting in decreased growth [Yavelow, J., Tuccillo, A., Kadner, S. S., Katz, J. and Finlay, T. H. “α₁-antitrypsin blocks the release of transforming growth factor-α from MCF-7 human breast cancer cells.” J. Clin. Endocrinol. Metab. 82, 745-752 (1997)]. In rats, administration of AT promoted hepatic fibrosis [Ozeki, T., Imanishi, K., Ueda, H., Uchiyama, T., Funakoshi, K., Suzuki, K., Ohuchi, K., Kan, M. and Satoh, T. “α₁-antitrypsin and hepatic fibrosis.” Br. J. Exp. Path. 70, 143-152 (1989)]. Finally, in monocytes, AT was found to modulate iron metabolism [Graziadei, I., Weiss, G., Egger, C., Niederwieser, D., Patsch, J. R. and Vogel, W. “Modulation of iron metabolism in monocytic THP-1 cells and cultured human monocytes by the acute-phase protein α₁-antitrypsin.” Exp. Hematol. 26, 1053-1060 (1998)]. None of these effects are related to the effects of AT on insulin sensitivity as described in the present application.

Several studies have demonstrated that the activity of AT can be inhibited by gemfibrozil (GF) [Janciauskiene, S. and Eriksson, S. “An interaction between Gemfibrozil and alpha₁-antitrypsin.” J. Internal Med. 236, 357-360 (1994)], hydrophobic bile acids [Janciauskiene, S. and Eriksson, S. “The interaction of hydrophobic bile acids with the α₁-proteinase inhibitor.” FEBS Lett. 343, 141-145 (1994)], and cholesterol [Janciauskiene, S. and Eriksson, S. “In vitro complex formation between cholesterol and α₁-proteinase inhibitor.” FEBS Lett. 316, 269-272 (1993)]. GF has been used in human therapy to decrease plasma triglyceride levels and enhance high-density lipoprotein cholesterol (“good” cholesterol) levels in order to prevent coronary heart disease [Yuan, J., Tsai, M. Y. and Hunninghake, D. B. “Changes in composition and distribution of LDL subspecies in hypertriglyceridemic and hypercholesterolemic patients during gemfibrozil therapy.” Atherosclerosis 110, 1-11 (1994); Schaefer, E. J., Lamon-Fava, S., Cole, T., Sprecher, D. L., Cilla, D. D., Balagtas, C. C., Rowan, J. P. and Black, D. M. “Effects of regular and extended-release gemfibrozil on plasma lipoproteins and apolipoproteins in hypercholesterolemic patients with decreased HDL cholesterol levels.” Atherosclerosis 127, 113-122 (1996); Rubins, H. B., Robins, S. J., Collins, D., Fye, C. L., Anderson, J. W., Elam, M. B., Faas, F. H., Linares, E., Schaefer, E. J., Schectman, G., Wilt, T. J., and Wittes, J. “Gemfibrozil for the secondary prevention of coronary heart disease in men with low levels of high-density lipoprotein cholesterol.” N. Engl. J. Med. 341, 410-418 (1999)].

The activity of AT is also known to be inhibited by small peptides whose sequence correspond to the unprimed (N-terminal) side of the active site which allows complex formation between the peptide and AT. The structure of these peptides of varying length is described by Schulze et al. [Schulze, A. J., Fronert, P. W., Engh, R. A. and Huber, R. “Evidence for the extent of insertion of the active site loop of intact α₁-proteinase inhibitor in β-Sheet A.” Biochemistry 31, 7560-7565 (1992)].

### SUMMARY OF THE INVENTION

In one embodiment, the present invention provides a method of treating or delaying the progression or onset of diabetes in subjects that exhibit above-normal blood levels of inflammation marker protein by administering an inhibitor of α₁-antitrypsin (AT). Inflammation marker proteins include α₁-antitrypsin, interleukin-6, and C-reactive protein. Suitable inhibitors of AT include gemfibrozil (GF) and active derivatives thereof, and lithocholic acid (LCA) and active derivatives thereof. Small peptide inhibitors of AT may also be suitable.

In another embodiment, the present invention provides a method of enhancing or restoring the sensitivity of mammals to the metabolic actions of insulin in subjects who exhibit above-normal blood levels of inflammation marker protein by administering an inhibitor of α₁-antitrypsin sufficient to enhance or restore the sensitivity of the subject to the metabolic actions of insulin. In still another embodiment, the present invention provides a method of enhancing or restoring the sensitivity of a diabetic human to the metabolic actions of insulin by administering an inhibitor of α₁-antitrypsin. In another embodiment, the present invention provides a treatment regimen for delaying the onset of diabetes in subjects who exhibit increased blood levels of inflammation marker protein by periodically administering an inhibitor of α₁-antitrypsin. The present invention also provides a treatment regimen for treating the progression of diabetes in subjects who exhibit increased blood levels of inflammation marker protein by administering an inhibitor of α₁-antitrypsin as needed.

The methods and treatment regimens may further include co-administering an anti-diabetic medicament. The anti-diabetic medicament may be insulin, an insulin secretogogue, a biguanide, an inhibitor of α-glucosidase, a thiazolidinedione, or combinations of these. The method may include further administering an anti-inflammatory agent. The subject for these methods may be a human or other mammal.

### BRIEF DESCRIPTION OF THE DRAWINGS

FIG. 1 shows a gel picture which indicates that purified AT, used in Examples 3-7, does not contain visible amounts of contaminating proteins as determined by Coommassie blue staining. Lane 2 represents the starting material (human placental alkaline phosphatase from Sigma-Aldrich, Inc.); lanes 3 and 4 demonstrate the presence of proteins after various purification steps; and lane 5 represents the final purified AT preparation as indicated by the arrow. Lane 1 contains molecular mass standards of 97 kDa (top), 66 kDa, 45 kDa, 31 kDa, and 22 kDa (bottom), in that order.

FIG. 2 demonstrates that in fasting (16 hours) C57/Black female mice, (i) intraperitoneal administration of 3 g/kg glucose elevated the blood glucose level about 1.6 to 2.2-fold during the subsequent 3-hour period, (ii) insulin initially strongly inhibited the rise in blood glucose level, and that (iii) intraperitoneal administration of commercial AT (1.5 mg/mouse) administered 24 hours prior to administration of insulin blocked the action of insulin. The insert shows that AT alone had no significant effect on the blood glucose level.

FIG. 3 demonstrates that in differentiated rat L6 muscle cells, insulin (▴) exerted more than two-fold stimulatory effects on the uptake of D-[¹⁴C]glucose (A) as well as the synthesis of [¹⁴C]glycogen (B) as compared to untreated cells (), and that 0.4 mg/ml of purified AT (▪) inhibited both insulin effects after incubations for 2 hrs or 6 hours, but not after 30 min incubation.

FIG. 4 indicates the concentration-dependent inhibitory effects of AT (0-400 μg/ml) on insulin-stimulated (▴) cellular uptake of D-[¹⁴C]glucose (A) as well as synthesis of [¹⁴C]glycogen (B) and [¹⁴C]total lipid (C) from radiolabeled glucose in differentiated L6 cells. AT in the absence of insulin (), had practically no effect.

FIG. 5 demonstrates that in differentiated L6 cells, both gemfibrozil (GF) and lithocholic acid (LCA) were capable of preventing the strong inhibitory effect of AT on insulin (Ins)-stimulated glycogen synthesis. Neither GF nor LCA had a significant effect in the absence of AT.

### DETAILED DESCRIPTION OF THE INVENTION

The following findings indicate that the methods and regimens of the invention may be therapeutically effective: (i) in humans, there is a correlation between increased synthesis of AT and decreased insulin sensitivity, (ii) in glucose tolerance tests performed using an appropriate mouse model, administration of AT decreases the effects of insulin on glucose metabolism, and (iii) administration of GF in vitro inhibits the activity of AT and reduces AT's inhibitory effect on insulin-stimulated glucose metabolism.

It has been found that AT synthesis and secretion is enhanced during inflammation, chronic and even sub-chronic inflammation as well as increased expression of AT are risk factors for Type 2 diabetes, and that administration of AT to mice was able to completely block the insulin action on blood glucose level. In glucose tolerance test, initial rise of blood glucose triggers insulin release from the islets, which then stimulates glucose metabolism. Blood sugar levels then decline within 30-60 minutes of glucose administration. In the experiments described herein, administration of AT had no clear effect alone on blood glucose level, but administration of AT delayed normalization of blood glucose level in glucose tolerance tests. This observation further indicates that AT is able to desensitize the action of insulin on the glucose metabolizing system.

Therefore, an appropriate level of inhibition of AT may be effective to reverse reduction in insulin sensitivity and thereby decrease the chances of developing Type 2 diabetes in a significant segment of population.

### Method for Treating or Delaying the Progression or Onset of Diabetes

In one embodiment, the present invention provides a method of treating or delaying the progression or onset of diabetes in a mammal by identifying a mammal with above-normal blood levels of AT, interleukin-6, or C-reactive protein (collectively referred to as “inflammation marker proteins”), and administering to the mammal a therapeutically effective amount of an inhibitor of α₁-antitrypsin.

The phrase “inhibitor of α₁-antitrypsin” denotes an agent that inhibits the activity of AT. It is not intended to refer to an agent that decreases the blood level of AT but does not inhibit the activity of AT, such as an agent that inhibits cellular synthesis of AT. As used herein, the phrases “inhibitor of AT” and “AT inhibitor” are used interchangeably to refer to an inhibitor of α₁-antitrypsin.

A “therapeutically effective amount” of an inhibitor of AT is targeted at attaining or maintaining a level of AT in the mammal's blood that is within the normal range for that mammal. The range of normal AT level for humans is about 1.3 mg AT/mL blood. A therapeutically effective amount of inhibitor of AT may be effective in increasing the sensitivity of the mammal to the metabolic actions of insulin. A therapeutically effective amount of inhibitor of AT may be effective to inhibit the activity of excess AT without compromising the basic physiological function of the remaining active AT.

Since development of insulin insensitivity and Type 2 diabetes are multi-factorial, there is a need for patient selection based on the blood levels of inflammation marker protein before administration of an AT inhibitor may be recommended. If determination of blood levels of inflammation marker protein repeatedly indicate chronic or sub-chronic inflammation, then administration of AT inhibitor is expected to improve insulin sensitivity in these patients.

Any of the tests to determine blood levels of AT, interleukin-6 and C-reactive protein are readily performed by technicians trained in the art using commercially available kits and reagents. Conventional or specially modified supplies such as chromatographic columns, electrophoresis gels, protein markers or stains, or immunoassay kits may be employed. For example, the level of AT in the blood of a subject can be accurately determined by a radial immunodiffusion of a sample of the subject's blood, such as by using a kit available from Behring Diagnostics (Mannheim, Germany). If the level of AT is found to be greater than the normal level of AT in the blood, which is about 1.3 mg AT per mL blood in humans, then administration of AT inhibitor is expected to improve insulin sensitivity.

Of the known inhibitors of AT, gemfibrozil (GF) has been the most studied. It is commercially available from Sigma-Aldrich, Inc. (St. Louis, Mo., USA). GF is used in humans to enhance the blood level of high density lipoprotein cholesterol and to decrease triglyceride level. Most regimens use 600-1200 mg GF once daily in an oral administration [Yuan, J., Tsai, M. Y. and Hunninghake, D. B. “Changes in composition and distribution of LDL subspecies in hypertriglyceridemic and hypercholesterolemic patients during gemfibrozil therapy.” Atherosclerosis 110, 1-11 (1994); Schaefer, E. J., Lamon-Fava, S., Cole, T., Sprecher, D. L., Cilla, D. D., Balagtas, C. C., Rowan, J. P. and Black, D. M. “Effects of regular and extended-release gemfibrozil on plasma lipoproteins and apolipoproteins in hypercholesterolemic patients with decreased HDL cholesterol levels.” Atherosclerosis 127, 113-122 (1996); Rubins, H. B., Robins, S. J., Collins, D., Fye, C. L., Anderson, J. W., Elam, M. B., Faas, F. H., Linares, E., Schaefer, E. J., Schectman, G., Wilt, T. J., and Wittes, J. “Gemfibrozil for the secondary prevention of coronary heart disease in men with low levels of high-density lipoprotein cholesterol.” N. Engl. J. Med. 341, 410-418 (1999)].

In none of these studies did GF exert any significant side effects, which makes it an attractive candidate for inhibiting in vivo the activity of excess AT that reduces insulin sensitivity. Thus, in subjects with high blood AT levels, daily administration of 600-1200 mg of GF appears to be safe. Data in the literature provide guidance with respect to the amount of GF that will inhibit the activity of excess AT without compromising the basic physiological function of the remaining active AT [Janciauskiene, S. and Eriksson, S. “An interaction between Gemfibrozil and alpha₁-antitrypsin.” J. Internal Med. 236, 357-360 (1994)].

The amount of GF required to be therapeutically effective can be measured in various ways. It has been shown that daily oral administration of GF at the 600 mg level inhibited AT activity by 30-60% [Janciauskiene, S. and Eriksson, S. “An interaction between Gemfibrozil and alpha1-antitrypsin.” J. Internal Med. 236, 357-360 (1994)]. This observation provides a benchmark from which to calculate the amount of GF which needs to be administered to restore AT activity to the normal level. By way of example only, if the blood AT level is 30% higher than normal, then daily administration of about 300 to 600 mg of GF is expected to be sufficient to normalize AT activity and to significantly decrease the risk for Type 2 diabetes. However, if AT activity is twice of the normal level, then 1,000-1,500 mg of GF per day may be initially needed to normalize the level of AT in the blood. Furthermore, by monitoring the level of AT in a subject's blood, the amount of GF administered may be varied to maintain a near normal blood level of AT.

Active derivatives of GF, lithocholic acid (LCA), and active derivatives of LCA may also be used as an AT inhibitor. As used herein, the phrase “active derivative” is used to refer to a derivative or substitute for the stated chemical species that operates in a similar manner to produce the intended effect, and is structurally similar and physiologically compatible. The inhibitors of AT may be administered in various ways including orally, intravascularly, intraperitoneally, subcutaneously, intramuscularly, intranasally, or topically, for example.

Small peptide inhibitors of AT may also be suitable to modulate blood levels of AT. Suitable small peptides are described, for example, by Schulze et al. [Schulze, A. J., Fronert, P. W., Engh, R. A. and Huber, R. “Evidence for the extent of insertion of the active site loop of intact α₁ proteinase inhibitor in β-Sheet A.” Biochemistry 31, 7560-7565 (1992)]. Since the active site loop, inhibited by the peptides, plays a critical role in the various biological actions of AT, it is reasonable to assume that these and similar inhibitory peptides will also prevent the effects of AT on blood sugar level.

The method may further include the step of co-administering to the mammal an anti-diabetic medicament. The anti-diabetic medicament may include insulin, an insulin secretogogue, a biguanide, a sulfonylurea, an inhibitor of α-glucosidase, a thiazolidinedione, human placental alkaline phosphatase, or a combination of these. Use of human placental alkaline phosphatase is described in U.S. application Ser. No. 10/317,916 (U.S. Pub. App. 2004/0115185) filed Dec. 12, 2002; U.S. application Ser. No. 10/441,992 (U.S. Pub. App. 2004/0120940) filed May 20, 2003; and PCT/US03/38838 (WO04/054609) filed Dec. 5, 2003.

These anti-diabetic medicaments are known treatments of diabetes and may be administered orally or by any other suitable method. The term “co-administering” indicates that the anti-diabetic medicament and inhibitor of AT are administered together (but not necessarily simultaneously) as part of a planned course of treatment intended to treat or delay the progression or onset of diabetes.

The method may also further include the step of administering to the mammal an anti-inflammatory agent. These agents are known in the art and may include non-steroidal anti-inflammatory drugs (“NSAIDs”), such as cyclooxygenase-2 inhibitors. Compounds that are cyclooxygenase-2 (“cox-2”) inhibitors and methods for the preparation of these compounds have been reported in the art.

Representative compounds that are commercially available include rofecoxib (4-[4-(methylsulfonyl)phenyl]-3-phenyl-2(5H)-furanone), marketed under the trade name VIOXX (Merck & Co., Inc., Whitehouse Station, N.J.), celecoxib (4-[5-(4-methylphenyl)-3-(trifluoromethyl)-1H-pyrazol-1-yl]), marketed under the trade name CELEBREX (G.D. Searle & Co., Chicago, Ill.), and valdecoxib (4-(5-methyl-3-phenyl-4-isoxazolyl)benzenesulfonamide), marketed under the trade name BEXTRA ((G.D. Searle & Co.). Other suitable anti-inflammatory agents include acetylsalicylic acid, ibuprofen, or active derivatives of these.

Administration of an anti-inflammatory agents may alter the amount of AT in the mammal. Therefore, if multiple administrations are given, the therapeutically effective amount of AT inhibitor may change to reflect the amount of AT in the mammal at the time the AT inhibitor is administered.

The method may be used to treat mammals that have been diagnosed as diabetic. It may also be used to delay the onset of diabetes in those mammals that are insensitive to the effects of insulin, but have not been diagnosed with diabetes. The method may be effective to reduce or maintain a human's blood glucose level to below about 10 mM, and preferably in the normal range of 4 mM to about 6 mM.

The method may also be used to prevent a mammal with Type 2 diabetes from progressing into Type 1 diabetes. If a mammal becomes less sensitive to insulin due to Type 2 diabetes, the β-cell islets initially produce and release more insulin to compensate for decreased insulin sensitivity. After continual increased production of insulin, the insulin secretion system will break down, and the mammal will develop Type 1 diabetes. By restoring the sensitivity of the mammal to insulin, the β-cell islets will produce normal amounts of insulin and are thus less likely to cease insulin production.

### Methods of Enhancing or Restoring Sensitivity to Insulin

In another embodiment, the present invention provides a method of enhancing or restoring the sensitivity of a mammal to the metabolic actions of insulin. The method includes the steps of identifying a mammal with an above-normal level of inflammation marker protein, and administering an inhibitor of AT sufficient to enhance or restore the sensitivity of the mammal to the metabolic actions of insulin.

In another embodiment, the invention provides a method of enhancing or restoring the sensitivity of a diabetic human to the metabolic actions of insulin. The method includes the steps of identifying a diabetic human with an above-normal level of inflammation marker protein, and administering a therapeutically effective amount of an inhibitor of AT sufficient to enhance or restore the sensitivity of the diabetic human to the metabolic actions of insulin.

The step of selecting the mammal or human with above-normal blood levels of inflammation marker protein may be done in the manner described above. The AT inhibitor may be gemfibrozil or an active derivative thereof, or lithocholic acid or an active derivative thereof, or a small peptide inhibitor, as described above. The AT inhibitor may be administered in the manner described above.

The methods may further include the step of co-administering an anti-diabetic medicament to the subject. The anti-diabetic medicaments may include the agents described above. The method may also further include the step of administering to the mammal an anti-inflammatory agent. The anti-inflammatory agents are described above.

The methods may be effective to reduce a human's blood glucose level to below about 10 mM, and preferably in the normal range of 4 mM to about 6 mM. The method may be used in mammals that are diabetic, or in mammals that have lower than normal sensitivity to insulin but have not been diagnosed diabetic.

### Treatment Regimen of Delaying Onset of Diabetes

In another embodiment, the present invention provides a treatment regimen for delaying the onset of diabetes in a mammal. The treatment regimen includes the steps of identifying a non-diabetic mammal with an above-normal level of an inflammation marker protein, and periodically administering to the mammal an inhibitor of AT.

The term “periodically” refers to repeated administration of AT inhibitor targeted to restoring or maintaining a normal about of AT in the mammal's blood. In this embodiment, the periods do not have to be uniform. The therapeutically effective amount of AT inhibitor may be different at each administration, depending upon the amount of AT or other inflammation marker protein present in the mammal.

The method may further include the step of co-administering an anti-diabetic medicament to the subject. The anti-diabetic medicaments may include the agents described above. The method may also further include the step of administering to the mammal an anti-inflammatory agent. The anti-inflammatory agents are described above.

### Treatment Regimen for Treating the Progression of Diabetes

The invention provides a treatment regimen for treating the progression of diabetes in a mammal. The treatment regimen includes the steps of identifying a diabetic mammal with above-normal blood levels of inflammation marker protein, and administering an AT inhibitor as needed.

The phrase “as needed” indicates that the therapeutically effective amount of AT inhibitor may be different at each administration, depending upon the amount of AT or other inflammation marker protein present in the mammal.

The method may further include the step of co-administering an anti-diabetic medicament to the subject. The anti-diabetic medicaments may include the agents described above. The method may also further include the step of administering to the mammal an anti-inflammatory agent. The anti-inflammatory agents are described above.

The method may further include the step of monitoring the level of AT in the mammal's blood. Monitoring the level of AT would generally be done on an ongoing basis. If the level of AT at any time is closer to an amount of AT that is normal for that mammal than when the mammal began the regimen, then the amount of AT inhibitor may be adjusted to reflect the amount of AT in the mammal's blood.

### Medicaments and Combinations Comprising an Inhibitor of α₁-Antitrypsin

The invention provides for the use of an inhibitor of α₁-antitrypsin for the manufacture of a medicament for treating or delaying the progression or onset of diabetes. The invention further provides for the use of an inhibitor of α₁-antitrypsin for the manufacture of a medicament for enhancing or restoring the sensitivity of a mammal to the metabolic actions of insulin. For either of these uses, the inhibitor of AT may be gemfibrozil or an active derivative thereof, lithocholic acid or an active derivative thereof, or a small peptide inhibitor of AT. The medicament may take a conventional form suitable for normal means of administration.

The invention further provides a combination of agents for simultaneous, separate, or sequential use for treating or delaying the progression or onset of diabetes, comprising a therapeutically effective amount of an inhibitor of α₁-antitrypsin and an anti-inflammatory agent. Also provided by the invention is a combination of agents for simultaneous, separate, or sequential use for treating or delaying the progression or onset of diabetes, comprising a therapeutically effective amount of an inhibitor of α₁-antitrypsin and an anti-diabetic medicament. The inhibitors of AT, anti-inflammatory agents, and anti-diabetic medicaments described above are suitably employed in the combinations. A combination of a therapeutically effective amount of an inhibitor of α₁-antitrypsin, an anti-inflammatory agent, and an anti-diabetic medicament is also within the scope of the invention.

### Methods and Kits for Identifying an Appropriate Subject

The invention also provides a method for identifying a subject in need of therapy for treating or delaying the progression or onset of diabetes by administration of an inhibitor of α₁-antitrypsin, comprising: a) measuring the level of an inflammation marker protein in a sample of the subject's blood; and b) determining whether the measured level is an above-normal blood level of the inflammation marker protein. In one embodiment, the inflammation marker protein is AT. By way of example, the range of normal AT level for humans is about 1.3 mg AT/mL blood; a level significantly higher than this would be considered “above normal.”

The invention also provides for the use of an assay kit for the identification of a subject in need of therapy for treating or delaying the progression or onset of diabetes by administration of an inhibitor of α₁-antitrypsin, wherein the use includes measuring the level of an inflammation marker protein in a sample of the subject's blood.

The step of measuring may be done in vitro in some embodiments. An immunoassay kit may be employed for measuring the level of an inflammation marker protein. The assay kit may be a radial immunodiffusion kit, for example. A Western blot analysis kit may also be suitable as the assay kit.

### EXAMPLES

In a first set of experiments performed using an appropriate mouse model, the desensitizing effect of α₁-antitrypsin on insulin-induced reduction of blood glucose levels was determined by classical glucose tolerance tests. In a second set of experiments, the desensitizing effect of AT on insulin-stimulated glucose metabolism in differentiated rat L6 muscle cells was confirmed. In a third set of experiments, two inhibitors of AT, gemfibrozil (GF) and lithocholic acid (LCA), were shown to prevent the desensitizing effect of AT on insulin-stimulated glycogen synthesis in differentiated L6 cells.

### Example 1

### Purification of AT

A partially purified human placental alkaline phosphatase preparation (PALP) was acquired from Sigma-Aldrich, Inc. AT is the major contaminant of the commercially obtained PALP. The PALP was first purified by successive concanavalin A-Sepharose and Q-Sepharose chromatography as described Chang et al. [Chang, T.-C., Huang, S.-M., Huang, T.-M. and Chang, G.-G., “Human placenta alkaline phosphatase: An improved purification procedure and kinetic studies.” Eur. J. Biochem. 209, 241-247 (1992)]. The Q-Sepharose fraction, which still contained placental alkaline phosphatase in addition to AT, was further purified to homogeneity by t-butyl HIC chromatography [Chang, T.-C., Huang, S.-M., Huang, T.-M. and Chang, G.-G., “Human placenta alkaline phosphatase: An improved purification procedure and kinetic studies.” Eur. J. Biochem. 209, 241-247 (1992)]. The 5 ml bed volume t-butyl HIC cartridge was connected to a Pharmacia FPLC system and the fractions containing AT were pooled. The purity was confirmed by sodium dodecyl sulfate polyacrylamide gel electrophoresis (SDS-PAGE) using Coommassie blue stain. The purified protein was identified as AT by sequence analysis. The sequence analysis was performed independently by the Mayo Clinic Protein Core Facility (Rochester, Minn., USA). The protein concentration was determined by the Lowry assay, using bovine serum albumin as standard, with a protein assay kit from Sigma-Aldrich, Inc. according to the instructions. This purification procedure has been previously published [She, Q.-B., Mukherjee, J. J., Crilly, K. S. and Kiss, Z. “α₁-antitrypsin can increase insulin-induced mitogenesis in various fibroblast and epithelial cell lines.” FEBS Lett. 473, 33-36 (2000)].

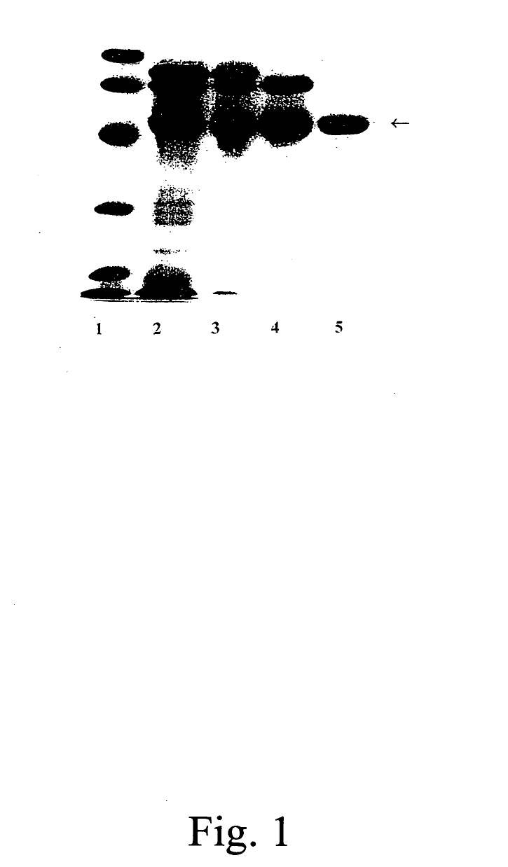

FIG. 1 shows a picture of a stained gel. The gel includes the commercially obtained partially purified placental alkaline phosphatase preparation (shown in lane 2), purified by successive Concanavalin A-Sepharose (lane 3), Q-Sepharose (lane 4), and t-butyl HIC chromatography using 2 M-0 M ammonium sulfate gradient (lane 5). Lane 1 contains molecular mass standards of 97 kDa (top), 66 kDa, 45 kDa, 31 kDa, and 22 kDa (bottom) in that order. FIG. 1 demonstrates that while the commercially obtained preparation contains three major proteins (one of them is AT as indicated by the arrow, while a ˜66 kDa band represents placental alkaline phosphatase) and several minor proteins, the purified preparation contains only AT. Identification of the AT band by sequence analysis was performed by the Mayo Clinic Protein Core Facility (Rochester, Minn., USA).

### Example 2

### Effect of Insulin and Commercial AT on Blood Glucose Levels in C57/Black Female Mice in Glucose Tolerance Tests

C57/Black female mice weighing 22-23 g were fasted for 16 hours. Four animals were used for each of four treatment groups. The first and fourth treatment group received intraperitoneal injections of 1500 μg commercially obtained AT. Exactly 23 hours and 45 minutes after the AT was administered to the first and fourth treatment group, the animals in the first and second treatment group received 0.5 I.U. of insulin. Exactly 15 minutes after the insulin was administered (i.e., 24 hours after the AT was administered to the first treatment group), glucose (3 g/kg) was injected intraperitoneally into the first, second and third treatment groups. Blood samples were taken from the eyes (canthus), and glucose concentrations in whole blood samples were immediately measured with the fast Glucose C test (Wako Chemicals USA Inc., Richmond, Va., USA). The data presented are the mean ±S.D. of four determinations, one with each animal.

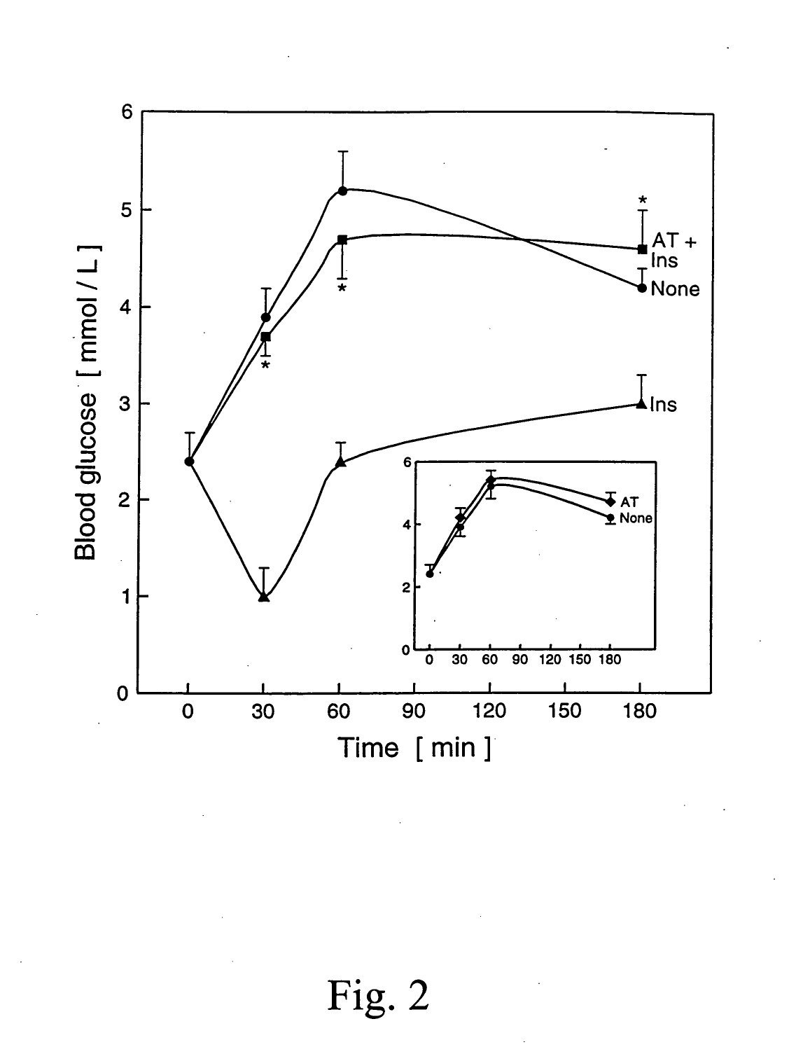

The results, shown in FIG. 2, demonstrate that in fasting mice, (i) intraperitoneal administration of 3 g/kg glucose elevated the blood glucose level about 1.6 to 2.2-fold during the subsequent 3-hour period, (ii) insulin initially strongly inhibited the rise in blood glucose level, and that (iii) prior (24 hours) intraperitoneal administration of purified AT (1.5 mg/mouse) blocked the action of insulin. Data presented in the insert in FIG. 2 shows that administration of AT alone to the fourth treatment group had no significant effect on the blood glucose level.

### Example 3

### Effect of Insulin and Purified AT on Blood Glucose Levels in C57/Black Female Mice in Glucose Tolerance Tests

C57/Black female mice weighing 23-25 g were fasted for 16 hours. Five animals were used for each of the three treatment groups. The first treatment group received intraperitoneal injections of 1200 μg purified AT, i.e. AT purified by the procedure of Example 1. Exactly 23 hours and 45 minutes after the AT was administered to the first treatment group, the animals in the first and second treatment group received 0.5 I.U. of insulin. Exactly 15 minutes after the insulin was administered (i.e., 24 hours after AT was administered to the first treatment group), glucose (3 g/kg) was injected intraperitoneally into the first, second and third treatment group. Blood samples were immediately measured with the fast Glucose C test (Wako Chemicals Inc. Richmond, Va., USA).

Data are given in Table 1 below. The data presented are the mean ±S.D. of five determinations, one with each animal.

TABLE 1
Determination of blood glucose levels for the experiment of Example 3.

| | Glucose level (mM) | | | | |
|---|---|---|---|---|---|
| Treatment | 0 min | 30 min | 60 min | 180 min | 360 min |
| None | 1.4 ± 0.3 | 4.0 ± 0.2 | 5.2 ± 0.3 | 4.4 ± 0.1 | 4.2 ± 0.3 |
| Insulin | 1.7 ± 0.2 | N.D.* | 2.5 ± 0.2 | 3.0 ± 0.3 | 3.3 ± 0.3 |
| AT + Insulin | 2.0 ± 0.3 | 3.9 ± 0.2 | 4.7 ± 0.2 | 4.6 ± 0.4 | 4.1 ± 0.3 |

*N.D means not detectable (below 1 mM).

The results demonstrate that in fasting (24 hours) C57/Black female mice (i) intraperitoneal administration of 3 g/kg glucose elevated the blood glucose level more than 3-fold by the 60^{th} minute, (ii) insulin initially strongly inhibited the rise in blood glucose level, and that (iii) intraperitoneal administration of highly purified AT (1.2 mg/mouse) administered 24 hours prior to administration of insulin blocked the action of insulin. Comparison of data obtained using commercial (Example 2) and purified (Example 3) AT preparations indicate no significant difference in the inhibitory effect on the action of insulin.

### Example 4

### Effect of Purified AT on Blood Glucose Levels in C57/Black Female Mice in Glucose Tolerance Tests

C57/Black female mice, weighing 22-25 g were fasted for 24 hours. Four animals were used for each of two groups. The first group received intraperitoneal injections of 1200 μg purified AT, i.e. AT purified by the procedure of Example 1. Exactly 24 hours after the AT was administered to the first group, glucose (3 g/kg) was injected intraperitoneally into mice in both groups. Blood samples were immediately measured with the fast Glucose C test (Wako Chemicals Inc. Richmond, Va., USA). The experiment was repeated a second time.

Data are given in Table 2 below. The data presented for each experiment are the mean ±S.D. of four determinations, one with each animal.

TABLE 2
Determination of blood glucose levels for the experiment of Example 4.

| | Glucose level (mM) | | | | |
|---|---|---|---|---|---|
| Treatment | 0 min | 30 min | 60 min | 120 min | 180 min |
| Experiment | | | | | |
| 1 | | | | | |
| None | 2.1 ± 0.4 | 8.3 ± 0.4 | 6.8 ± 0.5 | 3.4 ± 0.2 | 2.4 ± 0.3 |
| AT | 2.3 ± 0.3 | 8.5 ± 0.6 | 7.1 ± 0.5 | 6.1 ± 0.4 | 4.7 ± 0.5 |
| Experiment | | | | | |
| 2 | | | | | |
| None | 2.3 ± 0.4 | 7.4 ± 0.5 | 6.0 ± 0.6 | 3.5 ± 0.4 | 2.7 ± 0.3 |
| AT | 2.7 ± 0.3 | 7.9 ± 0.3 | 6.5 ± 0.5 | 5.9 ± 0.4 | 4.5 ± 0.4 |

The results demonstrate that in fasting (24 hours) C57/Black female mice (i) intraperitoneal administration of 3 g/kg glucose elevated the blood glucose level about 4-fold within 30 minutes, and (ii) while highly purified AT had no major effects in the first 60 min, it clearly delayed return of blood glucose level to near-normal level during the next 120 min.

In these experiments, the mice were fasted for 24 hours, instead of the 16-hour fasting period employed in Examples 2 and 3. The longer fasting period may explain why glucose injections resulted in higher initial blood glucose levels and more rapid decline in blood glucose level thereafter. Rapid disappearance of glucose indicated that the mice employed in experiments described in Example 4 were more sensitive to insulin and/or insulin production. In either case, AT delayed normalization of blood glucose level, which can only be explained by its interference with the action of insulin.

### Examples 5-7

### Effects of AT on Glucose Uptake and Metabolism in Differentiated Rat L6 Muscle Cells

Rat L6 skeletal muscle cells were obtained from the American Type Culture Collection, Rockville, Md. L6 cell monolayers were induced to differentiate in Minimal Essential Medium (MEM) containing 2% (v/v) fetal bovine serum (FBS) and 1% (v/v) antibiotic/antimycotic solution. The cells were fed fresh medium every 48 hours. Myoblast differentiation, which occurred by about the 7-9^{th} day, was monitored by phase contrast microscopy. Differentiated L6 cells are widely used models for studying glucose metabolism in the skeletal muscle. The present study was focused on these cells, because skeletal muscle accounts for about 70-80% of total blood glucose metabolism in the human body.

To examine cellular glucose uptake and metabolism, differentiated (attached) cells in 12-well plates were incubated at 37° C. in a CO₂ incubator (95% air: 5% CO₂) with D-[¹⁴C]glucose (1 μCi/mL) in 2% FBS-containing medium containing 5.5 mM unlabeled glucose for 0.5-6 hours as indicated at the corresponding Figures. At the termination of incubation, the medium was removed. An aliquot of the medium was taken to determine the loss of [¹⁴C]glucose from the medium which corresponds to its uptake by cells. Cells were washed twice with 3 ml medium to remove traces of medium containing unincorporated radioactivity. Ice-cold 99.8% methanol/0.2% water (v/v) mixture (1 ml) was then added to the monolayers and cells were extracted for 2 hours at −20° C. This resulted in the precipitation of glycogen and the solubilization of cellular free glucose and lipids. The methanol phase (1 ml) and a following 1 ml methanol wash were added to 2 ml chloroform followed by the addition of 3 ml water. Of the resulting two phases (separation was facilitated by brief centrifugation), the lower phase contained the total radiolabeled lipids, while the upper phase contained [¹⁴C]glucose.

Aliquots of the upper and lower phases were taken to scintillation vials to determine the amounts of radiolabeled glucose and total lipids, respectively, by liquid scintillation counting. Precipitated [¹⁴C]glycogen was suspended in 0.75 ml of 1 M NaOH and transferred to scintillation vial. This procedure was repeated with another 0.75 ml aliquot of NaOH. To neutralize the suspension, 10 mM HCl (approximately 150 μL) was then added to the vials, followed by the addition of 8.5 ml Ecolume (scintillation fluid). While the 1 M NaOH suspension contained some particulate material (mostly protein which was not labeled with radiolabeled glucose), no precipitate remained in the suspension after addition of Ecolume. This procedure resulted in quantitative removal of precipitated glycogen. No additional radioactivity could be removed from the well by neutralized 30% KOH.

In some experiments, a published procedure [Huang, D., Cheung, A. T., Parsons, J. T. and Bryer-Ash, M. “Focal adhesion kinase (FAK) regulates insulin-stimulated glycogen synthesis in hepatocytes.” J. Biol. Chem. 277, 18151-18160, (2002)] was used to determine glucose incorporation into glycogen in a method similar to the one described above. After incubations with [¹⁴C] glucose, cells were solubilized with 20% KOH for 2 hours. Lysates were extracted with 8% tricarboxylic acid, neutralized with 2.0 M HCl, then boiled for 5 min. The total glycogen was precipitated by the addition of 80% ethanol (final concentration) for 2 hours at −20° C. followed by centrifugation at 1100×g for 10 min. This step was repeated.

After the pellets had been re-dissolved in distilled water, the samples were precipitated again using the same method described above, and yielded essentially the same results. In subsequent experiments the initial methanol precipitation method described above was used because it allowed for analysis of cellular glucose, glycogen and lipids from the same sample.

### Example 5

### Time-Dependent Effects of AT on Insulin-Stimulated Glucose Uptake and Glycogen Synthesis in Differentiated L6 Cells

Three groups of differentiated L6 cells, incubated in 12-well plates in 2% FBS-containing medium, were treated as follows. A first control group () was untreated. A second group (▴) was treated with 500 nM insulin for 30 min. A third group (▪) was treated first with 0.4 mg/ml of AT followed by an addition of 500 nM insulin for 30 min. Following the addition of D-[¹⁴C]glucose, each group was incubated in the continuous presence of insulin and AT, as described above, for 0.5-6 hours.

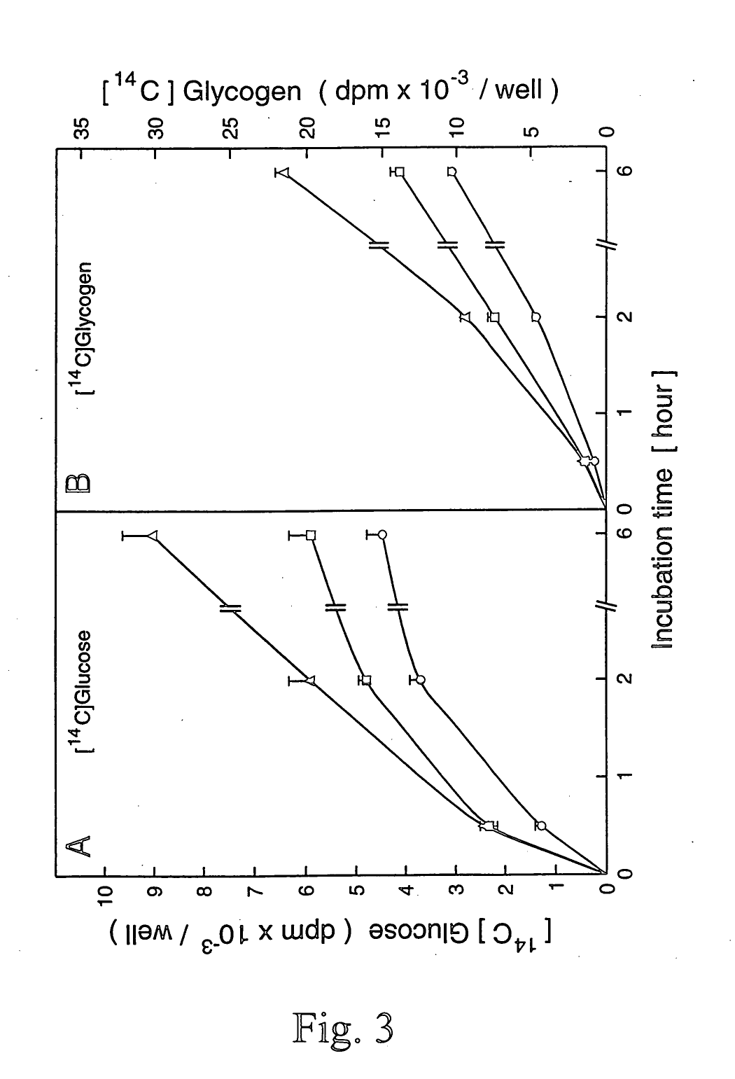

For each group, the amounts of cellular radiolabeled glucose (FIG. 3A) and glycogen (FIG. 3B) were determined by liquid scintillation counting. The data are the mean ±S.D. of three independent incubations in one experiment (the experiment was repeated once with similar results). Compared to the control group (), the cellular levels of both free [¹⁴C]glucose (FIG. 3A) and [¹⁴C]glycogen (FIG. 3B) were approximately doubled in the presence of insulin after incubations for 2-6 hours (▴). The addition of 0.4 mg/ml of AT inhibited the insulin effect by approximately 40% to 60% after incubation for 6 hours (▪). AT had no such inhibitory effects over a shorter (30 min) incubation time.

### Example 6

### Concentration-Dependent Inhibitory Effects of AT on Insulin-Stimulated Glucose Metabolism in Differentiated L6 Cells

Four groups of differentiated L6 cells, incubated in 12-well plates in 2% FBS-containing medium, were treated as follows. A first control group was untreated. A second group was treated with 500 nM insulin for 30 min. A third group was treated with 0.05 to 0.4 mg/ml of AT for 10 min. Finally, a fourth group was treated with 0.05 to 0.4 mg/ml of AT for 10 min followed by the addition of 500 nM insulin for 30 min. Following the addition of D-[¹⁴C]glucose, each group was then incubated in the continuous presence of insulin and AT for 5 hours.

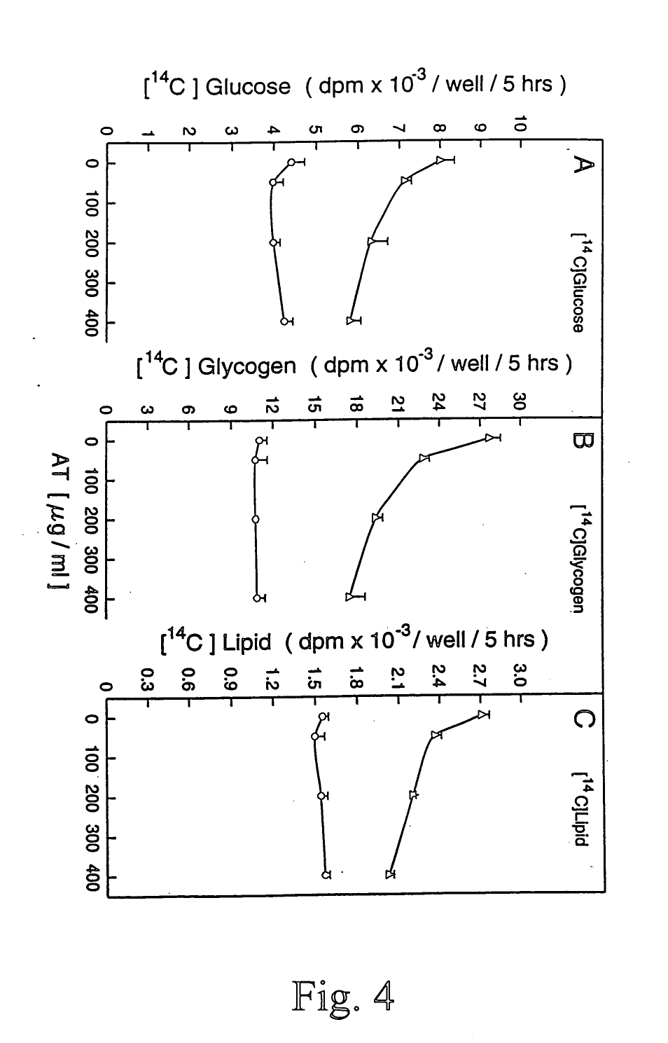

For each group, the amounts of cellular radiolabeled glucose (FIG. 4A), glycogen (FIG. 4B), and total lipid (FIG. 4C) were determined by liquid scintillation counting. The data are the mean ±S.D. of three independent incubations in one experiment (the experiment was repeated once with similar results). In the absence of insulin (), the presence of AT did not significantly affect the cellular levels of any of these radiolabeled metabolites. However, in the presence of insulin (▴), 0.2 to 0.4 mg/ml AT decreased the cellular levels of [¹⁴C] glucose (FIG. 4A) as well as the synthesis of cellular [¹⁴C] glycogen (FIG. 4B) and [¹⁴C]total lipid (FIG. 4C) from [¹⁴C] glucose by at least 50%.

### Example 7

### Gemfibrozil (GF) and Lithocholic Acid (LCA) Prevent the Inhibitory Effect of AT on Insulin-Stimulated Synthesis of Glycogen in Differentiated L6 Cells

Three groups of differentiated L6 cells, incubated in 12-well plates in 2% FBS-containing medium were treated with insulin and AT as follows. A first control group was untreated. A second group was treated with 500 nM insulin for 30 min. A third group was treated with 0.4 mg/ml of AT for 10 min, followed by the addition of 500 nM insulin for 30 min. Within each group were sub-groups where either no other compound was added (□), or 25 mM GF was added to the cells 60 min prior to AT (□), or 50 μM LCA (▪) was added to the cells 60 min prior to treatment with AT. Then, following the addition of D-[¹⁴C]glucose, each group was incubated in the continuous presence of the above agents, as applicable, for 5 hours.

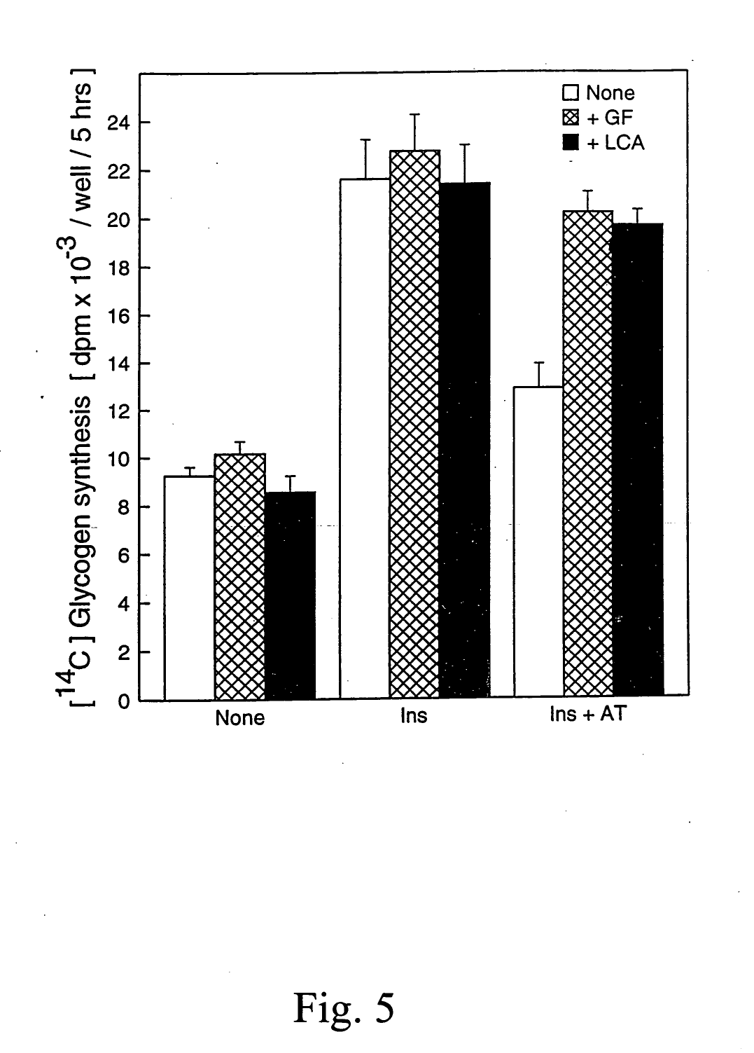

For each sub-group, the amount of cellular [¹⁴C]glycogen was determined by liquid scintillation counting. The data are the mean ±S.D. of four independent incubations in one experiment (the experiment was repeated once with similar results). As shown in FIG. 5, insulin stimulated the synthesis of [¹⁴C]glycogen about two-fold, while the presence of AT inhibited the insulin effect by about 70%. In the presence of either GF or LCA, AT had no such inhibitory effect.

This invention may take on various modifications and alterations without departing from the spirit and scope thereof. Accordingly, it is to be understood that this invention is not to be limited to the above-described, but it is to be controlled by the limitations set forth in the following claims and any equivalents thereof. It is also to be understood that this invention may be suitably practiced in the absence of any element not specifically disclosed herein.

In describing preferred embodiments of the invention, specific terminology is used for the sake of clarity. The invention, however, is not intended to be limited to the specific terms so selected, and it is to be understood that each term so selected includes all technical equivalents that operate similarly.

1. A method of treating or delaying the progression or onset of diabetes in a mammal comprising:
(a) identifying a mammal with an above-normal blood level of an inflammation marker protein; and
(b) administering to the mammal a therapeutically effective amount of an inhibitor of α₁-antitrypsin.

2. The method of claim 1 wherein the step of identifying includes performing an immunoassay, a Western blot analysis, or a chromatographic separation of a sample of the mammal's blood.

3. The method of claim 1 wherein the mammal is a human.

4. The method of claim 3 wherein the method is effective to maintain the human's blood glucose level below 10 mM.

5. The method of claim 3 wherein the method is effective to maintain the human's blood-glucose level between 4 mM and 6 mM.

6. The method of claim 1 wherein the inflammation marker protein is α₁-antitrypsin, C-reactive protein, interleukin-6 or a combination thereof.

7. The method of claim 1 wherein the inflammation marker protein is α₁-antitrypsin, the mammal is a human, and the above-normal blood level is greater than 1.3 mg α₁-antitrypsin/mL blood.

8. The method of claim 1 wherein the inhibitor of α₁-antitrypsin is gemfibrozil or an active derivative thereof.

9. The method of claim 8 wherein the mammal is a human, and wherein the therapeutically effective amount of gemfibrozil is about 300 to 1500 mg per day.

10. (canceled)

11. The method of claim 8 wherein gemfibrozil is administered daily, and the amount of gemfibrozil is adjusted daily to maintain a normal blood level of α₁-antitrypsin.

12. The method of claim 8 wherein gemfibrozil is administered once per day.

13. The method of claim 1 wherein the inhibitor of α₁-antitrypsin is lithocholic acid or an active derivative thereof.

14. The method of claim 1 wherein the method further comprises the step of:
(c) co-administering to the mammal an anti-diabetic medicament.

15. The method of claim 14 wherein the anti-diabetic medicament includes insulin.

16. The method of claim 14 wherein the anti-diabetic medicament comprises an insulin secretogogue, a biguanide, an inhibitor of α-glucosidase, a thiazolidinedione, or a combination thereof.

17. The method of claim 14 wherein the anti-diabetic medicament comprises human placental alkaline phosphatase.

18. The method of claim 1 wherein the method further comprises the step of:
(d) administering to the mammal an anti-inflammatory agent.

19. The method of claim 18 wherein the anti-inflammatory agent is a non-steroidal anti-inflammatory drug, acetylsalicylic acid, ibuprofen, or an active derivative thereof.

20. The method of claim 18 wherein the anti-inflammatory agent is a cyclooxygenase-2 inhibitor.

21. A method of enhancing or restoring the sensitivity of a mammal to the metabolic actions of insulin comprising:
(a) identifying a mammal with an above-normal blood level of an inflammation marker protein; and
(b) administering to the mammal an inhibitor of α₁-antitrypsin sufficient to enhance or restore the sensitivity of the mammal to the metabolic actions of insulin.

22.-116. (canceled)
